(19) United States
(12) Patent Application Publication
(10) Pub. No.: US 2010/0004568 A1
(43) Pub. Date: Jan. 7, 2010

(54) Treatment Device for a Device for Producing Shock Waves

(76) Inventors: Werner Schwarze, Jena (DE); Hugo Stephan, Jena (DE)

Correspondence Address: Scott R. Foster;Pandiscio & Pandiscio, 470 Totten Pond Road, Waltham, MA, 02451-1914, US

(21) Appl. No.: 12/226,303

(22) PCT Filed: May 10, 2007
(86) PCT No.: PCT/EP2007/004156

§ 371 Date: May 27, 2009

(30) Foreign Application Priority Data
May 13, 2006 (DE) 10 2006 022 417.5

(51) Int. Cl. A61B 17/225

(52) U.S. Cl. 601/4

(57) ABSTRACT

The invention relates to a treatment device (1) for a device (2) for producing shock waves (4) that are especially suitable for medical use. Said device comprises a housing (3), in which the device (2) for producing shock waves (4) is arranged, a focusing device (5) in which the housing (3) is inserted and which is sealed from the exterior in a liquid-tight manner by a membrane (10), a bellows structure or the like, a liquid (6) with which the focusing device (5) is filled and which encloses the device (2) for producing shock waves (4), and a conduit (7) through which the liquid (6) can be supplied to and discharged from the focusing device (5). The aim of the invention is to allow gases which naturally occur in the water or gas which is produced by the shock wave production to be chemically bound or to be reconverted to water. For this purpose it is e.g. necessary that as few water molecules as possible escape from the water circuit and are therefore present as reactants for the oxygen molecules for producing water. This aim is achieved by connecting the conduit (7) to a container (11) in which the liquid (6) can be chemically degassed in such a manner that the oxygen molecules (17) present in the liquid (6) react with hydrogen molecules (16) to form water (6) or react with a reducing agent.

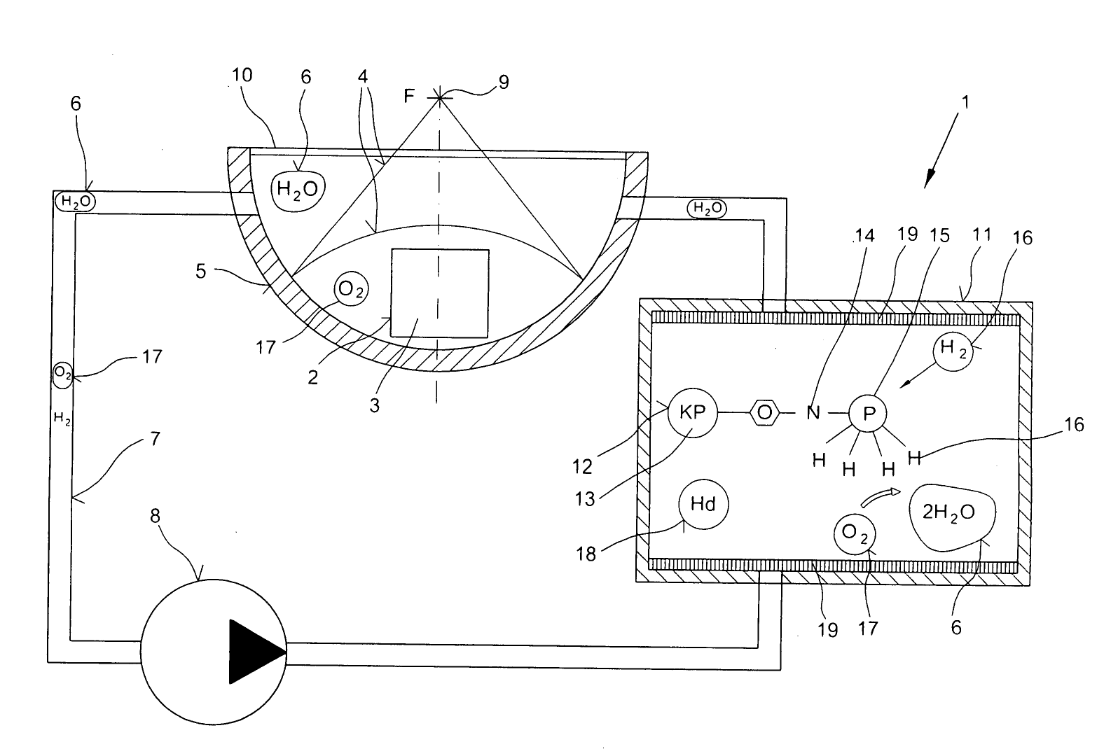

The invention relates to a device for producing and focusing shock waves, especially for medical use in accordance with the precharacterizing clause of patent claim 1.

Devices of this kind have been used for decades in medicine, for example in urology, as lithotripsers, in orthopedics for treating non-healing bone fractures or for insertion tendonitis or quite generally for promoting wound healing. In order to destroy kidney stones, for example, located in human organs from the outside of the body using acoustic shock waves, it is necessary to generate a large number of shock waves and to focus them so that the kidney stones are arranged in a particular area of the shock waves.

The shock waves are generated by various electrohydraulic, electromagnetic or piezoelectric systems, preferably in water. The reason for this is because the shock waves have to be decoupled and transferred into the body receiving treatment after they have been produced, and that the soft tissue of the body that they have to pass through has similar acoustic properties to water. At the same time, the water serves as a means of conducting away the process heat and for rinsing away the electrode erosion during generation of the shock wave. Furthermore, it is possible to couple to different body shapes and thicknesses by means of a coupling membrane and different water pressures in the membrane.

An electrode pair is used in the device, for example, to generate the shock wave, with the shock waves being generated between the electrodes by spark discharge. Furthermore, it is necessary to accommodate the electrodes in water because the water serves as a means of conduction and coupling for the shock waves. The situation for electromagnetic or piezoelectric systems is similar. Normally, the focusing device that aligns the shock waves is configured as a spherical reflector or as a system of lenses. Water is also provided in the focusing device as a means of conduction and coupling, in order to conduct the shock waves further from the focusing device. The focusing device is sealed by a membrane or a bellows in the direction of the treatment center to provide a liquid-tight seal, with the result that the water remains within the focusing device.

However, gases that occur in the atmosphere, namely O₂, N₂ and CO₂ are dissolved naturally in the water used. The ratio between the dissolved O₂ and N₂ gases in the water at room temperature is about 4 to 1, with the proportion of the dissolved CO₂ being negligible, meaning that the dissolved oxygen accounts for the highest percentage. If the water circuit is open or not sealed against diffusion of these gases, the water in the circuit will absorb gas repeatedly over time as well. In addition, oxygen and hydrogen are liberated during electrohydraulic shock wave generation due to shock ionization when the spark jumps. Even if the hydrogen escapes from the system by diffusion, the oxygen remains in the system in dissolved or gaseous form.

The dissolved gases, meanwhile, impede shock wave propagation because they lead to cavitation bubbles in the tension wave portion of the shock waves. It takes energy to create these bubbles and the microscopic gas bubbles in the acoustic field impede shock wave propagation due to dispersal and reflection.

As a result, a reduction in the efficiency of the device is measurable. Special degassing devices are installed in the water circuit in order to avoid this loss. Most of these function by heating, applying a negative atmospheric pressure, spraying the water into the negative atmospheric pressure or also by using semi-permeable membranes. Following this, the degassed water is guided back to the focusing device. All of these systems are complicated, expensive, bulky and require special pumping systems, have to be gas-tight and only result in values of greater than 1.0 to 1.5 micrograms of dissolved O₂ per liter of water. The target values are significantly lower, for example 1.0 micrograms of dissolved O₂ per liter of water.

For these reasons, it is normal for the water circuit to be degassed by negative pressure. Accordingly, the water flows out of the conduit into a container in which the oxygen molecules are removed from the water by negative pressure. Following this, the degassed water in channeled back into the focusing device through the conduit.

It has proven to be a disadvantage with negative pressure degassing that the container has to be made gas-tight in order to be able to generate a sufficiently powerful negative pressure inside the container so that the oxygen molecules can escape from the water. However, a container configuration of this kind is extremely cost-intensive to manufacture. Furthermore, the entire system has to be made gas-tight.

The purpose of the present invention is therefore to provide a treatment device of the kind mentioned in the introduction, by means of which the gases naturally occurring in the water or the gas resulting from the shock wave generation can be chemically bound or converted back into water. To achieve this, for example, it is necessary for as few hydrogen molecules as possible to escape from the water circuit which means they remain present as reaction partners for the oxygen molecules for recombining to make water.

This purpose is achieved by the features that are listed in the precharacterizing clause of patent claim 1.

Other advantageous further embodiments are disclosed on the subordinate claims.

The principal opportunity for removing the naturally occurring dissolved oxygen by chemical means involves reduction reactions. For example, sodium sulfite can be converted into sodium sulfate by absorbing oxygen. Both salts are soluble in water and non-toxic, therefore they are suitable for use in medical systems.

Another embodiment in accordance with the present invention uses a buffer medium in the water to prevent the hydrogen molecules created due to the shock wave generation from escaping. The buffer medium can be dissolved or suspended in the water, or can be present in the water circuit in the form of a reaction vessel through which the water flows.

The buffer medium binds the freely mobile hydrogen molecules present in the water and therefore keeps them in the water, which means the hydrogen molecules remain available to function as reaction partners for the freely mobile oxygen molecules. The sequence of chemical reactions this entails results in the hydrogen and oxygen molecules combining to make water.

In addition, the container does not have to be made gas-tight and sealed, because all that happens inside it is a chemical reaction between the hydrogen molecules and the oxygen molecules, which thereby reduces the manufacturing costs of the container.

In order to provide the free oxygen molecules with sufficient reaction partners in the form of hydrogen molecules, it is possible to add hydrazine or aqueous organic or inorganic acids to the water circuit. These substances continuously liberate hydrogen molecules into the fluid flowing into the container, with the effect that there are sufficient reaction partners available for the oxygen molecules present in the liquid.

The single drawing shows a sample embodiment configured in accordance with the present invention, the details of which are explained below. The FIGURE shows a treatment device for a device for generating shock waves and for a focusing device in which the generation device is inserted and into which water is filled as a transmission medium for the shock waves, as well as a water circuit formed by a conduit for treating the water in a container.

A treatment device 1 that is used for chemical degassing of water 6 should be assigned to a device 2 for generating shock waves 4. The device 2 for generating the shock waves 4 consists of a housing 3 in which the shock waves 4 are generated by electrode pairs (not illustrated) by means of voltage breakdown in the water. The shock waves 4 are propagated to the outside by means of a transmission and coupling medium in the form of water 6. As a result, both the housing 3 and the volume surrounding the housing 3 is completely filled with water 6.

To prevent the water 6 filled in the focusing device 5 from flowing out of it, the focusing device 5 is sealed by means of a membrane 10 in the direction of the treatment centre 9. The outside of the membrane 10 is coupled to the body to be treated. As is known, the predominant proportion of this body is water, so that the shock waves 4 propagate through the human body practically unimpeded.

As well as the natural initial gas load of the water represented by dissolved atmospheric gases, it has furthermore been shown that the electrochemical reaction occurring in the housing during electrohydraulic shock wave generation in the form of shock ionization liberates free hydrogen and oxygen molecules 16 and 17. The hydrogen molecules 16 diffuse into the atmosphere within a very short time without being impeded whereas the oxygen molecules 17 form gas bubbles and collect in the water 6 and even on the membrane 10, the focusing device 5 or the housing 3, thereby impeding the propagation of the shock waves 4.

To prevent this deposition of gases, in particular of oxygen molecules 17, the water 6 and therefore also the oxygen molecules 17 are removed from the focusing device 5 by means of a conduit 7. A pump 8 is provided in the line 7 by means of which the volumetric flow for circulating the water can be adjusted.

The conduit 7 emerges in a container 11 into which the water 6 therefore flows. Chemical degassing should be completed inside the container 11 to convert the oxygen molecules 17 with hydrogen into water 6. Therefore, a plurality of a buffer medium 12 is filled into the container 11 which are represented schematically. The buffer medium 12 consists, for example, of a synthetic resin pearl 13 to which a palladium particle 15 with a metalized surface is attached by means of a nitrogen bond 14. The freely mobile hydrogen molecules 16 or substances such as hydrazine or acids that liberate hydrogen flow past the palladium particles 15 and are bound by them.

This therefore results in three chemical reactions between the metalized outer layer of the palladium particles 15 and the hydrogen molecules 16 present in the water 6:

H₂+PdO→PdO+2H₂ 1st

As soon as the oxygen molecules 17 present in the water 6 flow past the hydrogen molecules 16 bonded to the palladium particles 15, a further chemical reaction takes place, namely:

2H+O—O→2HO—OH,

and this compound reacts further according to the following chemical formula

2H+2HO—OH→2H₂O.

This reaction sequence means that water 6 has been reformed from the hydrogen and oxygen molecules 16 and 17 respectively that were liberated by the shock ionization.

As soon as the water 6 in the container 11 has been chemically degassed, it is directed back into the focusing device 5 through the conduit 7.

What is claimed is:

1. A treatment device (1) for a device (2) for producing shock waves for medical use, with the device (2) comprising a housing (3) in which the device (2) for generating the shock waves (4) is arranged, and a focusing device (5) in which the housing (3) is disposed and which is sealed to the outside by means of a membrane (10) to provide a liquid-tight seal, with a liquid (6) filled in the focusing device (5) enclosing the device (2) for generating the shock waves (4), and with a conduit (7) through which the liquid (6) can be supplied to the focusing device (5) and can flow out of the focusing device (5), wherein the conduit (7) is connected to a container (11) in which the liquid (6) can be chemically degassed, and wherein oxygen molecules (17) present in the liquid (6) react with at least one of hydrogen molecules (16) to make water, and a reduction agent.

2. The treatment device in accordance with claim 1, wherein a plurality of buffer media (12) is filled in the container (11), by means of which the hydrogen molecules (16) are bound.

3. The treatment device in accordance with claim 2, wherein each of the buffer media (12) consists of synthetic resin pearls (13) with nano or microparticle structures on their surfaces with one or more palladium particles (15) attached thereto by means of at least one nitrogen bonds (14).

4. The treatment device in accordance with claim 3, wherein the palladium particles (15) have a metalized surface onto which the hydrogen molecules (16) can be chemically locked.

5. The treatment device in accordance with claim 1, wherein a chemical substance (18) is added to the container (11) and the hydrogen molecules (16) are transferrable from the substance (18) to the liquid (6) flowing through the container (11).

6. The treatment device in accordance with claim 5, wherein the chemical substance (18) comprises selected one of hydrazine and dilute acids.

7. The treatment device in accordance with claim 2, wherein insides of the container (11) in the area of inlet and outlet openings of the conduits (7) are covered by a selected one of a mat (19), a membrane, and a screen, which cannot be penetrated by the buffer media (12) and the hydrogen molecules (16).

8. The treatment device in accordance with claim 2, wherein at least one of the focusing device (5) and the conduit (7) is provided with a barrier layer made of the buffer media (12), which is impermeable to the hydrogen molecules (16).

9. The treatment device in accordance with claim 1, wherein a pump (8) is arranged in the conduit (7) by means of which the liquid (6) can be pumped through the container (11).

10. The treatment device in accordance with claim 1, wherein both the reduction agent and a reaction product are water soluble.

11. The treatment device in accordance with claim 1, wherein the reduction agent is present in the focusing device (5) and the container (11).

12. The treatment device in accordance with claim 1, wherein the reduction agent is a non-toxic substance.

13. The treatment device in accordance with claim 1, wherein the reduction agent is sodium sulfite.
